(19) United States
(12) Patent Application Publication
(10) Pub. No.: US 2023/0165984 A1
(43) Pub. Date: Jun. 1, 2023

(54) STERILIZATION BAG

(71) Applicant: OXTI CORPORATION, New Taipei City (TW)

(72) Inventor: Chih-Feng Ho, New Taipei City (TW)

(21) Appl. No.: 17/537,495

(22) Filed: Nov. 30, 2021

(51) Int. Cl. A61L 2/10; A61L 2/26

(52) U.S. Cl. CPC A61L 2/10; A61L 2/26; A61L 2202/26; A61L 2202/11; A61L 2202/121; A61L 2202/16

(57) ABSTRACT

A sterilization bag includes: a bag body, provided with an accommodation space and opening, the opening in communication with the accommodation space, and the accommodation space provided with at least one optical surface; and a ultraviolet light source, configured in the accommodation space, allowing the ultraviolet light source to irradiate the at least one optical surface. Whereby, the ultraviolet light source can disinfect and sterilize the accommodation space of the bag body to eliminate viruses and bacteria in the air or on an item, thereby having an epidemic prevention effect.

### TECHNICAL FIELD OF THE INVENTION

The present invention relates to a bag, and more particularly to a sterilization bag capable of disinfection and sterilization to have an epidemic prevention effect.

### DESCRIPTION OF THE PRIOR ART

For example, Taiwan Patent No. M603299 discloses a multifunctional storage bag, including a main storage bag and a plurality of sub storage bags, where the main storage bag is a pleated structure and is provided with a plurality of pleated surfaces, the inside of the main storage bag is configured with the sub storage bags; the sub storage bags are configured on the inside of the pleated surface. Furthermore, a male-female coupling element is arranged between the main storage bag and each of the sub storage bag. Whereby, the above multifunctional storage bag can be used for epidemic prevention by separating, cleaning or replacing its sub storage bags.

The conventional bag disclosed by the above patent achieving the purpose of epidemic prevention merely relies on separation use, cleaning or replacement, and the effect thereof is limited; viruses and bacteria can not be effectively eliminated.

### SUMMARY OF THE INVENTION

To overcome the above disadvantages, the present invention proposes a sterilization bag, including: a bag body, provided with an accommodation space and opening, the opening in communication with the accommodation space, and the accommodation space provided with at least one optical surface; and a ultraviolet light source, configured in the accommodation space, allowing the ultraviolet light source to irradiate the at least one optical surface,

wherein the ultraviolet light source is arranged inside a protective bracket with gaps or transparent protective container,

wherein the optical surface is composed of a plurality of adjacent polygonal blocks, and the optical surface is a reflective surface or refractive surface,

wherein the ultraviolet light source includes at least one ultraviolet light emitting diode (UV LED),

and wherein the ultraviolet light source is provided with a signal receiver.

With the above structure, the sterilization bag of the present invention can disinfect and sterilize the accommodation space of the bag body through the ultraviolet light source to eliminate viruses and bacteria in the air or on the item, thereby having an epidemic prevention effect. In addition, the ultraviolet light source may be arranged inside the protective bracket with gaps or transparent protective container, and the high temperature influence of the ultraviolet light source on the bag body can be prevented through the heat isolation protection function of the protective bracket or protective container. Furthermore, the remote controller and the signal receiver configured on the ultraviolet light source can be used to remotely control the on or off of the ultraviolet light source.

### BRIEF DESCRIPTION OF THE DRAWINGS

FIG. 1 is a schematically perspective view of a sterilization bag of the present invention;

FIG. 2 is a schematically perspective view of the sterilization bag of the present invention, in which an optical surface is composed of a plurality of adjacent polygonal blocks;

FIG. 3 is a schematically perspective view of the sterilization bag of the present invention, in which a ultraviolet light source is configured inside a protective bracket with gaps;

FIG. 4 is a schematically perspective view of the sterilization bag of the present invention, in which the ultraviolet light source is configured inside a transparent protective container;

FIG. 5 is a schematically perspective view of the sterilization bag of the present invention, in which remote control is implemented;

FIG. 6 is a schematically perspective view of the sterilization bag of the present invention, in which an item is placed in the accommodation space of a bag body from an opening; and

FIG. 7 is a schematically perspective view of the sterilization bag of the present invention, in which the ultraviolet light source is disinfecting and sterilizing the accommodation space of the bag body.

### DETAILED DESCRIPTION OF THE PREFERRED EMBODIMENTS

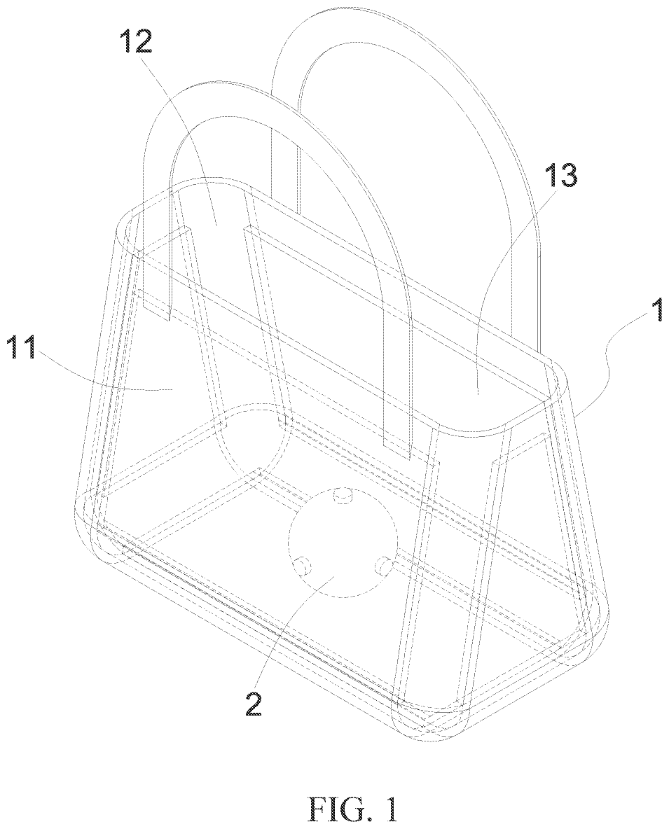

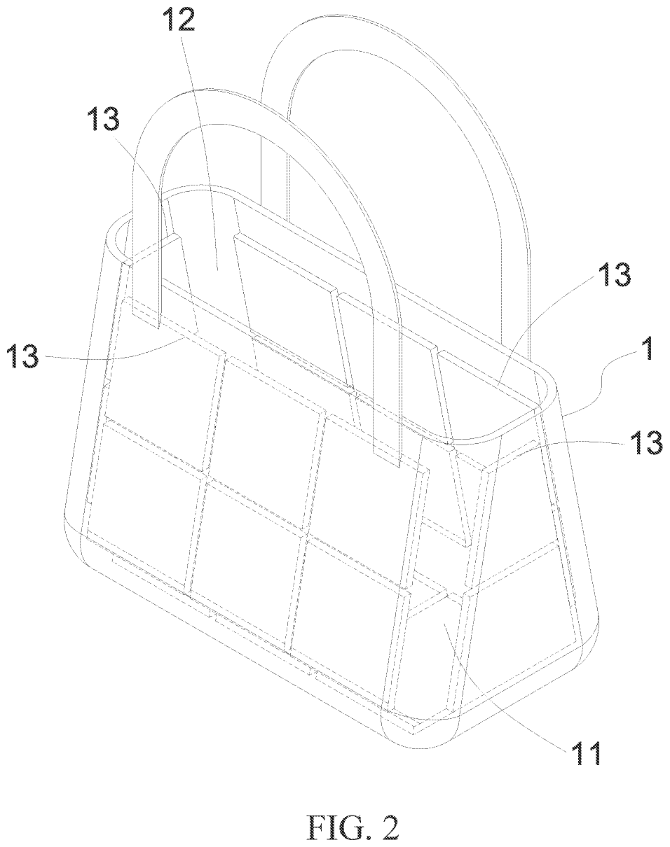

Referring to FIGS. 1 and 2, a sterilization bag of the present invention includes a bag body 1 and ultraviolet light source 2.

The bag body 1 is provided with an accommodation space 11 and an opening 12 in communication with the accommodation space 11. Furthermore, the accommodation space 11 is provided with at least one optical surface 13. The optical surface 13, as FIG. 2 shows, may be constituted by a plurality of adjacent polygonal blocks and may be a reflective surface or refractive surface, which can be achieved by means of reflective paint or refractive paint. In addition, the optical surface can also be composed of a smaller reflective surface and a smaller refractive surface to form a larger optical surface 13. Thus, light can be adjusted through the optical surface 13. For example, the reflective surface is used to concentrate the light so that the light illuminates a specific position in the accommodating space 11 in a concentrated manner, or the refractive surface is used to diverge the light and deflect the light to the outside of the bag body 1.

The ultraviolet light source 2 is configured in the accommodation space 11, allowing the ultraviolet light source 2 to illuminate the at least one optical surface 13. The ultraviolet light source 2 is used to provide disinfecting and sterilizing ultraviolet light and may include at least one ultraviolet light emitting diode (LED). Furthermore, the ultraviolet light source is in connection with a power source that supplies power.

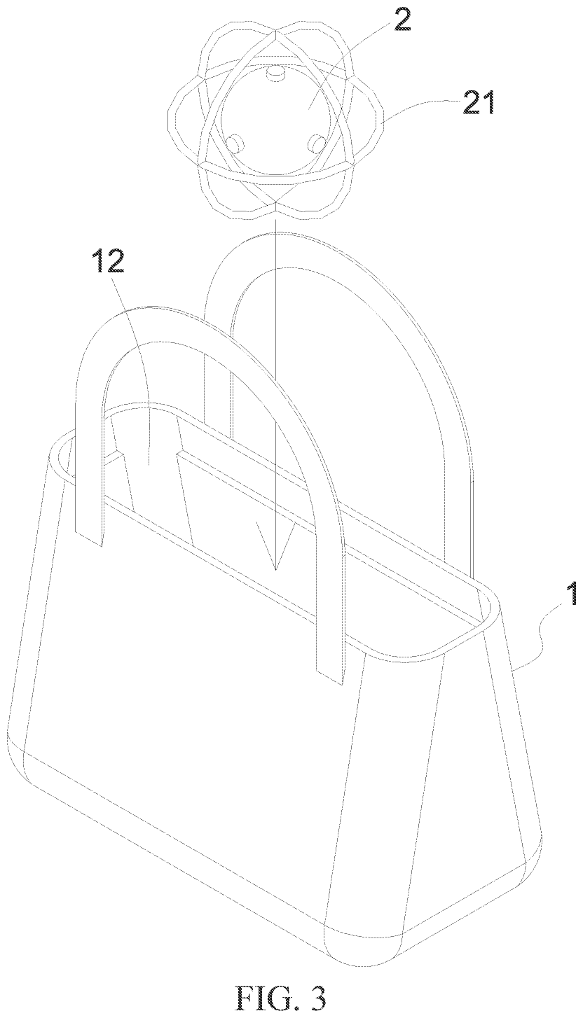

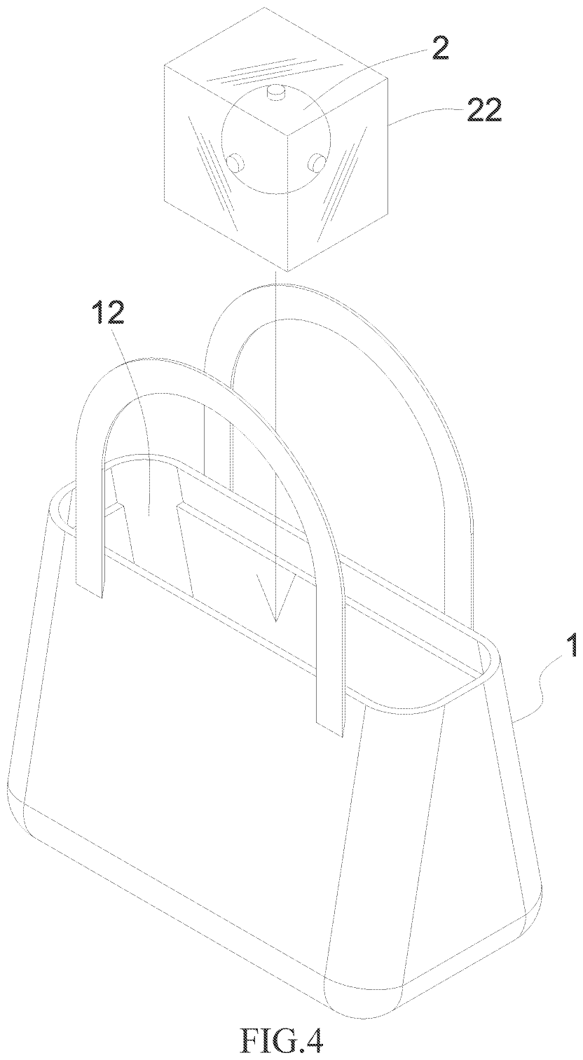

Referring to FIGS. 3 and 4, the ultraviolet light source 2 may be arranged inside a protective bracket 21 with gaps or a transparent protective container 22, through the heat isolation protective function of which the bag body 1 can be prevented from the high temperature influence of the ultraviolet light source 2, where the protective bracket 21 may be made of a plurality of steel wires or iron wires.

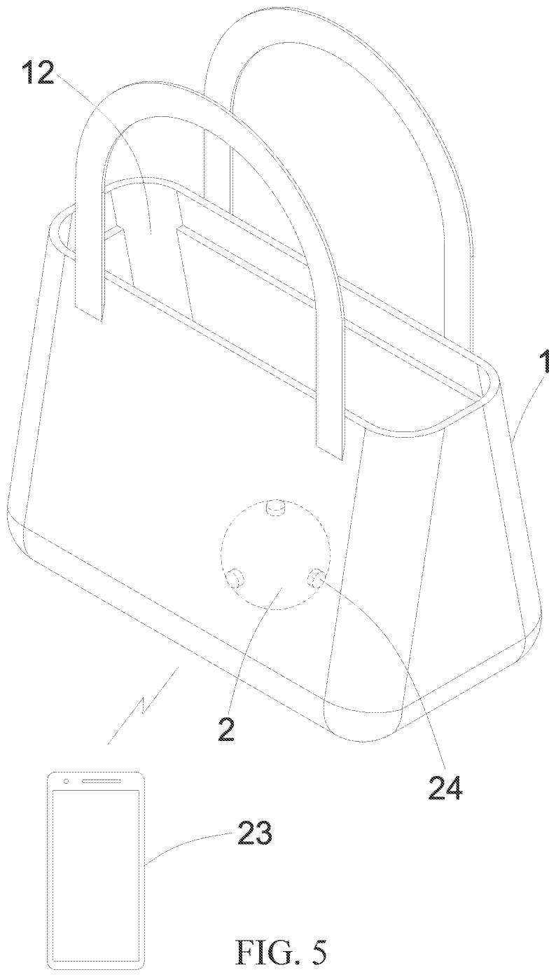

Referring to FIG. 5, the ultraviolet light source 2 is provided with a signal receiver 24 adapted to receive a radio signal, where the signal receiver 24 may, for example, such as a radio signal receiver, and the radio signal may come from a remote controller 23, thereby remotely controlling the on or off of the ultraviolet light source 2. A user may generate the radio signal through, for example, the application (APP) of a smart phone or a web browser, and transmit the radio signal to the signal receiver 24 via the internet or in other transmission manners.

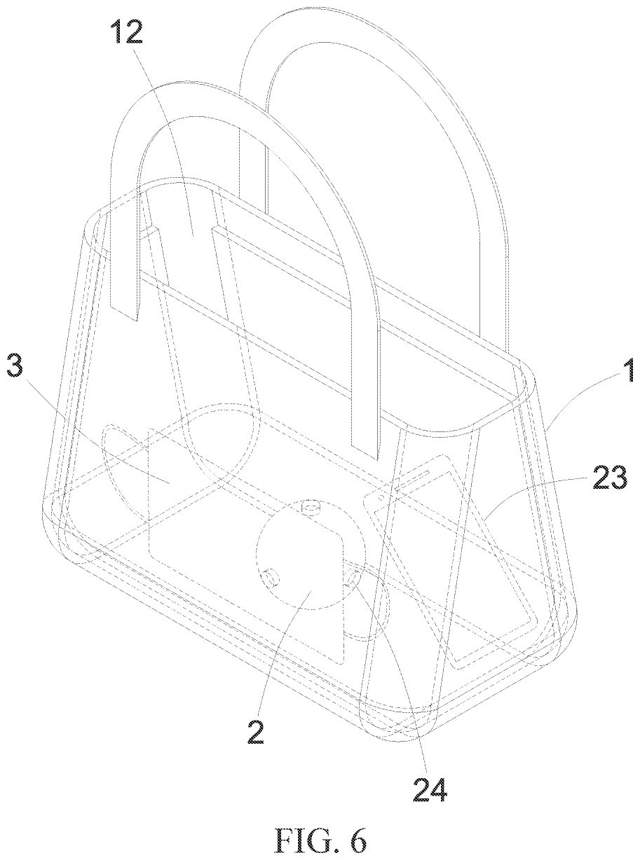

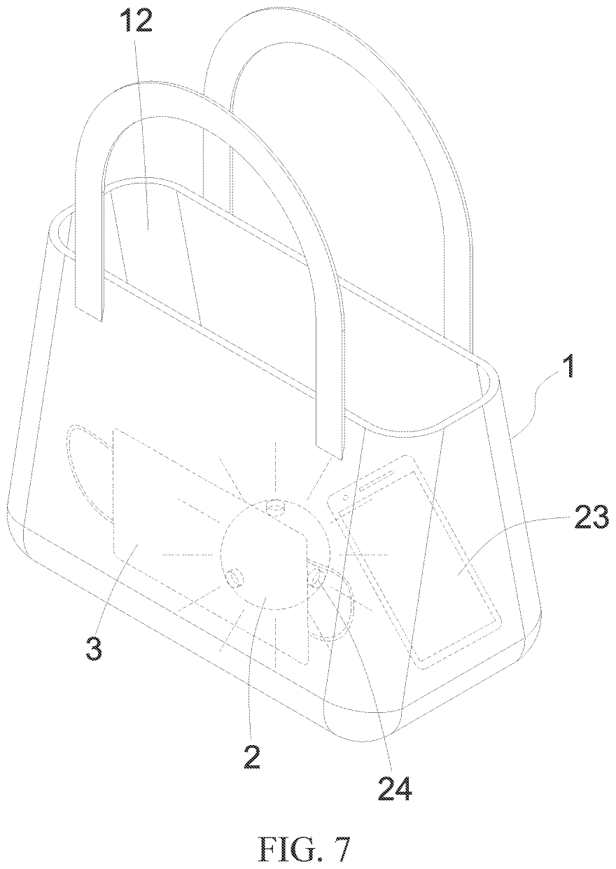

Referring to FIGS. 1 and 6, an item 3 can be placed in the accommodation space 11 of the bag body 1 through the opening 12. Referring to FIG. 7, the accommodation space 11 of the bag body 1 is disinfected and sterilized through the ultraviolet light source 2 to eliminate viruses and bacteria in the air or on the item 3, thereby having an epidemic prevention effect. For example, the ultraviolet light source 2 is relatively located at the left side of the item 3, and the ultraviolet light still can be reflected by the optical surface 3 (the reflective surface) and radiated indirectly onto the right side of the item 3 so that the dead angle irradiated by the ultraviolet light source 2 can be effectively reduced and at the same time the configuration number of the ultraviolet light sources 2 can also be reduced. Only one ultraviolet light source 2 is required to cooperate with the optical surface 13 to achieve an irradiation range without any dead angle. Furthermore, for example, it is also possible to diverge the ultraviolet light through the optical surface 13 (refracting surface) and deflect the ultraviolet light to the outside of the bag body 1, so as to adjust the light intensity in the bag body 1.

Therefore, the sterilization bag of the present invention can disinfect and sterilize the accommodation space 11 of the bag body 1 through the ultraviolet light source 2 to eliminate viruses and bacteria in the air or on the item 3. At the same time, the ultraviolet light source 2 may be arranged inside the protective bracket 21 with gaps or transparent protective container 22, and the high temperature influence of the ultraviolet light source 2 on the bag body 1 can be prevented through the heat isolation protection function of the protective bracket 21 or protective container 22. Furthermore, the remote controller 23 and signal receiver 24 can be used to remotely control the on or off of the ultraviolet light source 2.

I claim:

1. A sterilization bag, comprising:
a bag body, provided with an accommodation space and opening, said opening in communication with said accommodation space, and said accommodation space provided with at least one optical surface; and
a ultraviolet light source, configured in said accommodation space, allowing said ultraviolet light source to irradiate said at least one optical surface.

2. The sterilization bag according to claim 1, wherein said ultraviolet light source is arranged inside a protective bracket with gaps or transparent protective container.

3. The sterilization bag according to claim 1, wherein said optical surface is composed of a plurality of adjacent polygonal blocks, and said optical surface is a reflective surface or refractive surface.

4. The sterilization bag according to claim 1, wherein said ultraviolet light source comprises at least one ultraviolet light emitting diode (UV LED).

5. The sterilization bag according to claim 1, wherein said ultraviolet light source is provided with a signal receiver.
